(19) United States
(12) Patent Application Publication
(10) Pub. No.: US 2010/0099897 A1
(43) Pub. Date: Apr. 22, 2010

(54) STABLE ANHYDROUS CRYSTALLINE DOCETAXEL AND METHOD FOR THE PREPARATION THEREOF

(75) Inventors: Namdu Kim, Hwaseong-si (KR); Woo Seob Shin, Hwaseong-si (KR); Jaehyuk Jung, Goyang-si (KR); Gi Jeong Kim, Suwon-si (KR); Seung Hwan Cho, Busan (KR); Eun Jung Lim, Osan-si (KR); Youngho Moon, Suwon-si (KR); Young-Kil Chang, Seoul (KR); Gwan Sun Lee, Seoul (KR)

Correspondence Address: SUGHRUE MION, PLLC, 2100 PENNSYLVANIA AVENUE, N.W., SUITE 800, WASHINGTON, DC, 20037, US

(73) Assignee: HANMI PHARM. CO., LTD., Hwaseong-si, Gyeonggi-do (KR)

(21) Appl. No.: 12/532,887

(22) PCT Filed: Apr. 10, 2008
(86) PCT No.: PCT/KR08/02014

§ 371 Date: Sep. 24, 2009

(30) Foreign Application Priority Data
Apr. 10, 2007 (KR) 10-2007-0035065

(51) Int. Cl. C07D 305/14

(52) U.S. Cl. 549/510

(57) ABSTRACT

The present invention provides a stable anhydrous crystalline docetaxel which has anti-tumor and anti-leukemia activity, and method for the preparation thereof.

### FIELD OF THE INVENTION

The present invention relates to stable anhydrous crystalline forms of docetaxel and method for the preparation thereof.

### BACKGROUND OF THE INVENTION

Docetaxel is a potent anti-tumor chemotherapeutic agent having a broad spectrum of anti-tumor and anti-leukemia activity, which has been approved as a commercially marketable therapeutic agent against ovarian cancer and breast cancer.

There have been recorded three major crystal forms of docetaxel: docetaxel trihydrate (a); docetaxel hemihydrate (b); and anhydrous docetaxel (c) whose powder X-ray diffraction spectra are shown in FIG. 1 (see, U.S. Pat. No. 5,723,635 and [Zaske L., Perrin M.-A., Leveiller F. J. Phys. IV France 11, Pr10-221 (2001)]). The docetaxel trihydrate form is currently marketed for commercial use.

U.S. Pat. No. 5,723,635 discloses a method for preparing docetaxel trihydrate using a mixture of methyl isobutyl ketone, acetone and water. However, this method requires the use of a special procedure, centrifugal partition chromatography.

In addition, U.S. Pat. No. 6,022,985 discloses a method for preparing docetaxel trihydrate by dissolving docetaxel in ethanol, dropwisely adding water to the resulting solution at 50° C. to induce crystallization and drying the crystallized docetaxel crystal for 48 hrs at 38° C. and 80% relative humidity under a pressure of 5.07 kPa. Also, U.S. Pat. No. 6,838,569 discloses a method for preparing docetaxel trihydrate by dissolving docetaxel in acetonitrile, dropwisely adding water to the resulting solution at 68° C. to induce crystallization and drying the crystallized docetaxel crystal for 36 hrs at 36° C. under a reduced pressure of 650 torr.

The above-mentioned methods have problems in that the residual solvent remaining in the final product is difficult to remove, and the content of the 7-epimer, i.e. 4-acetoxy-2α-benzoyloxy-5-β, 20-epoxy-1,7α,10β-trihydroxy-9-oxo-tac-11-en-13-α-yl(2R,3S)-3-t-butoxycarbonylamino-2-hydroxy-3-phenylpropionate is in the range of 0.4 to 0.8%, which must be further purified to meet the purity requirement that the 7-epimer content be 0.5% or less.

Therefore, the present inventors have endeavored to develop an anhydrous crystalline form of docetaxel having the 7-epimer content of 0.1% or less, which is non-hygroscopic and stable under a high temperature/humidity condition.

### SUMMARY OF THE INVENTION

Accordingly, it is an object of the present invention to provide a stable anhydrous crystalline docetaxel and method for the preparation thereof.

In accordance with one aspect of the present invention, there is provided an anhydrous crystalline form of docetaxel of formula (I):

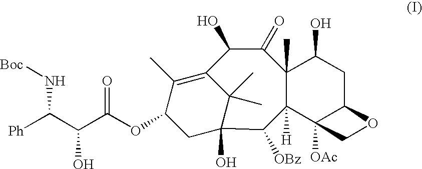

wherein,

Ph is phenyl;

Ac is acetyl;

Bz is benzoyl; and

Boc is t-butoxycarbonyl.

In accordance with another aspect of the present invention, there is a method of preparing the compound of formula (I), which comprises the steps of:

(i) dissolving docetaxel in an organic solvent;

(ii) adding an anti-solvent to the resulting solution; and

(iii) recovering the resulting crystals.

### BRIEF DESCRIPTION OF DRAWINGS

The above and other objects and features of the present invention will become apparent from the following description of the invention taken in conjunction with the following accompanying drawings, which respectively show:

FIG. 1: Powder X-ray diffraction spectra of docetaxel trihydrate (a), docetaxel hemihydrate (b), and anhydrous docetaxel (c); and

FIGS. 2 to 5: Powder X-ray diffraction spectra of the anhydrous crystalline docetaxel forms A, B, C and D, respectively.

### DETAILED DESCRIPTION OF THE INVENTION

The anhydrous crystalline docetaxel of the present invention, which comprises 0.1% or less of 7-epimer and is non-hygroscopic, and stable under a high temperature/humidity condition, is suitable for use in treating tumor and leukemia.

The anhydrous crystalline docetaxel of the present invention can be prepared by dissolving docetaxel in an organic solvent; adding an anti-solvent to the resulting solution to induce crystallization; recovering the resulting crystals by filtration, and drying the docetaxel crystals under a reduced pressure.

The anhydrous crystalline form of docetaxel of the present invention may vary depending on the preparation procedure. According to the present invention, the anhydrous crystalline form of docetaxel of the present invention may be any one of anhydrous crystalline docetaxel forms A, B, C and D.

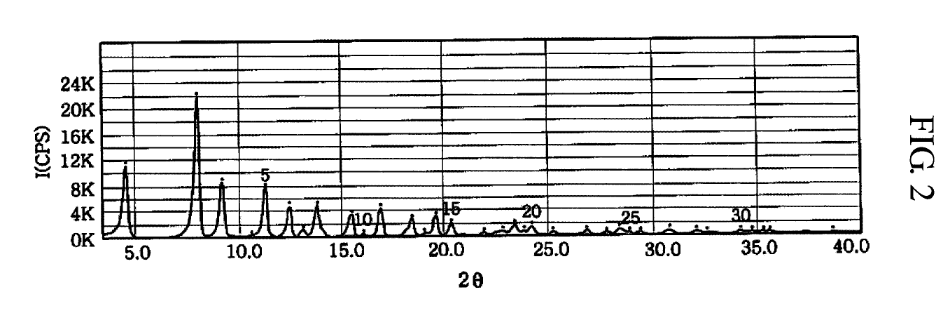

Specifically, in accordance with one aspect of the present invention, the X-ray diffraction spectrum of the anhydrous crystalline docetaxel form A shows major peaks having relative peak intensity (100×I/I₀; I: the peak intensity; I₀: the peak intensity of the maximum peak) of at least 56% at diffraction angles (2θ±0.1) of 4.64, 8.04, 9.24, 11.34, 12.54, 13.86, 15.52, 16.92, 18.48, 19.64, 20.40, 23.36, and 24.20 (see, Table 1 and FIG. 2).

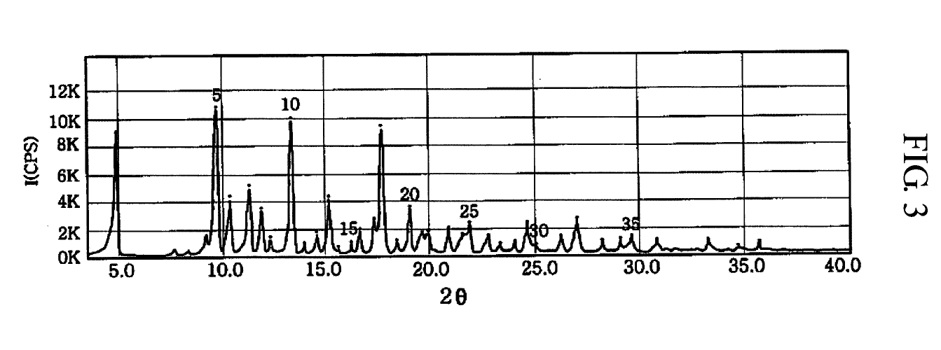

In accordance with another aspect of the present invention, the X-ray diffraction spectrum of the anhydrous crystalline docetaxel form B shows major peaks having relative peak intensity (100×I/I₀; I: the peak intensity; I₀: the peak intensity of the maximum peak) of at least 100% at diffraction angles (2θ±0.1) of 4.88, 9.22, 9.72, 10.38, 11.30, 11.88, 13.34, 14.56, 15.14, 16.62, 17.28, 17.66, 19.02, 19.62, 19.86, 20.86, 21.86, 24.58, and 26.98 (see, Table 2 and FIG. 3).

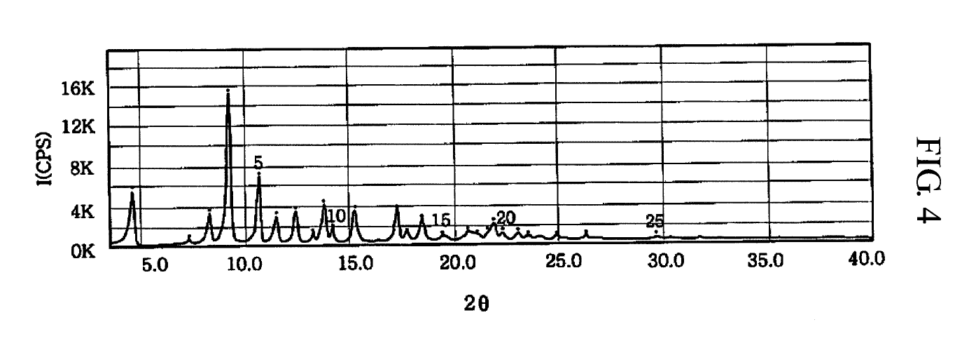

In accordance with still another aspect of the present invention, the X-ray diffraction spectrum of the anhydrous crystalline docetaxel form C shows major peaks having relative peak intensity (100×I/I₀; I: the peak intensity; I₀: the peak intensity of the maximum peak) of at least 55% at diffraction angles (2θ±0.1) of 4.62, 8.22, 9.20, 10.64, 11.44, 12.42, 13.80, 14.20, 15.28, 17.28, 18.46, 20.62, and 21.86 (see, Table 3 and FIG. 4).

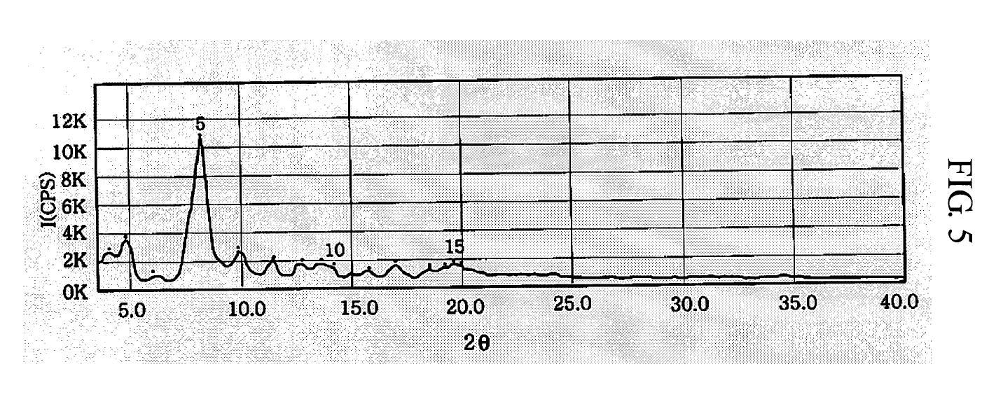

In accordance with a further aspect of the present invention, the X-ray diffraction spectrum of the anhydrous crystalline docetaxel form D shows major peaks having relative peak intensity (100×I/I₀; I: the peak intensity; I₀: the peak intensity of the maximum peak) of at least 50% at diffraction angles (2θ±0.1) of 4.06, 4.82, 7.58, 8.20, 9.84, 11.44, 12.76, 13.62, 14.16, 16.98, 19.18, 19.60, and 19.90 (see, Table 4 and FIG. 5).

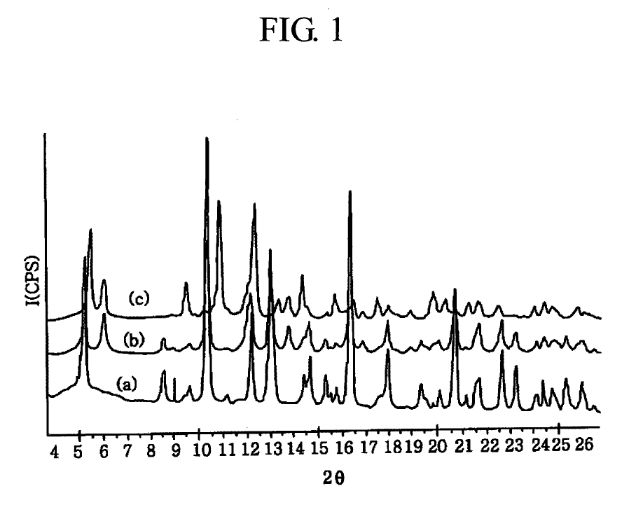

The X-ray diffraction patterns of the anhydrous crystalline docetaxel forms A, B, C, and D, which are shown in FIGS. 2 to 5, respectively, are each distinctively different from that of the anhydrous docetaxel prepared by conventional method shown in FIG. 1-a. Also, the inventive anhydrous crystalline docetaxel exhibits markedly improved storage stability: for example, it does not undergo any significant degradation during a long term storage under a high temperature/humidity condition (temperature: 60±2° C. and relative humidity: 75±5%).

Docetaxel used as the starting material in the present invention may be prepared by the procedure shown in Reaction Scheme (I). The procedure comprises the steps of:

(i) allowing (2R,3S)—N-t-butoxycarbonyl-4-phenylisoserin methylester of formula (2) to react with 1-dimethoxymethyl naphthalene in an organic solvent in the presence of an acid catalyst to obtain the oxazolidine methyl ester derivative of formula (3), and hydrolyzing the compound of formula (3) in the presence of a base to obtain the oxazolidine acid derivative of formula (4);

(ii) subjecting the compound of formula (4) to a coupling reaction with the protected 10-deacetylbaccatin of formula (5) in a solvent in the presence of a condensation agent to obtain the oxazolidine side chain-bearing taxane of formula (6);

(iii) reacting the compound of formula (6) in an organic solvent in the presence of an acid to obtain the docetaxel of formula (7) having protected 7- and 10-hydroxy groups; and

(iv) removing the protected 7- and 10-hydroxy groups from the compound obtained in (iii).

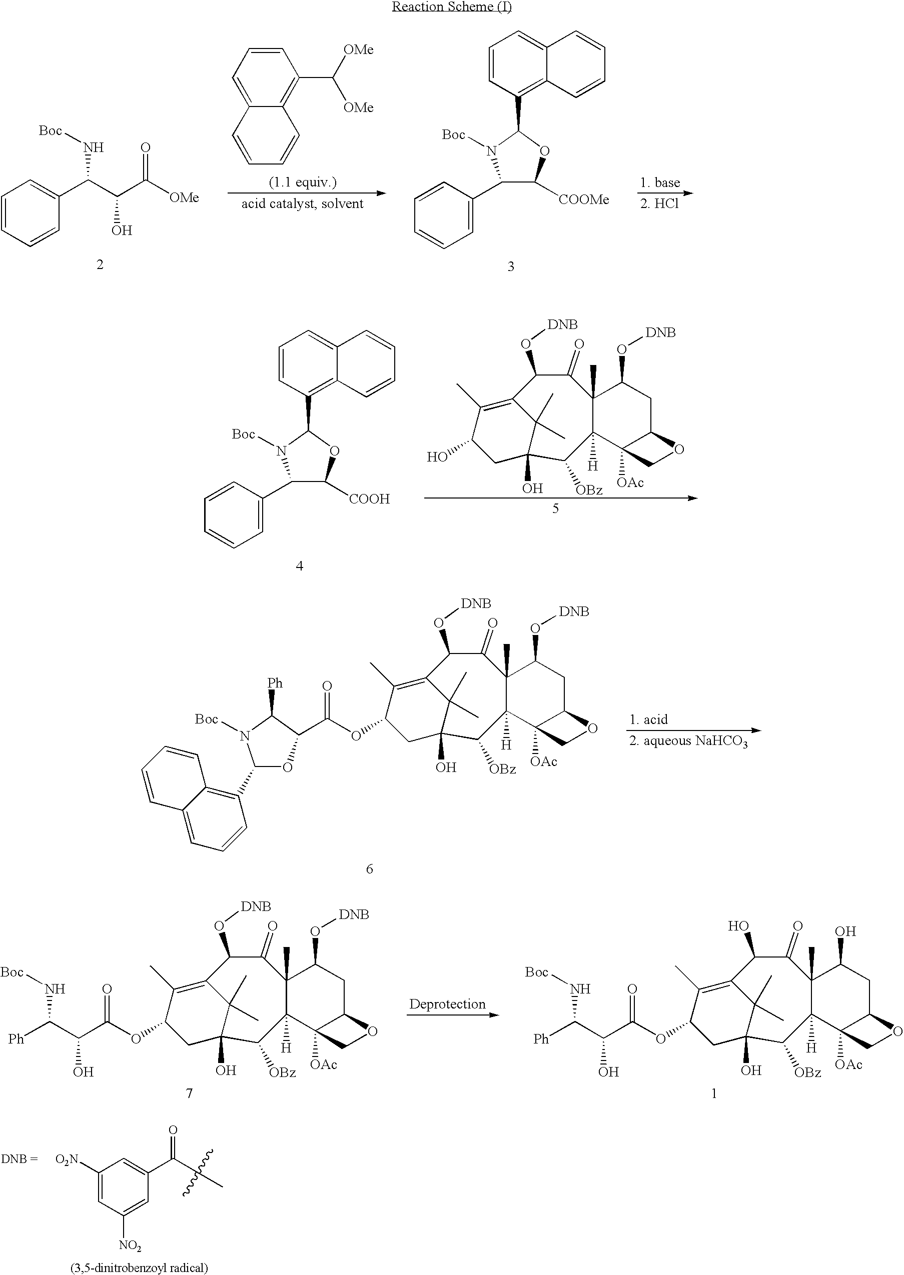

The anhydrous crystalline form of docetaxel prepared by the method of the present invention may vary depending on the solvent used in the reaction. Also, the anhydrous crystalline docetaxel of the present invention has a high purity of 98% or higher, which comprises the 7-epimer impurity in an amount of less than 0.1%.

The organic solvent used in dissolving docetaxel may be an ether such as diethyl ether, diisopropyl ether or tetrahydrofuran; an ester such as ethyl acetate or methyl acetate; a ketone such as methyl ethyl ketone; a mixture of dichloromethane and methanol; a mixture of dichloromethane and acetonitrile. Preferably, the amount of the organic solvent used in the inventive reaction is in the range of 5 to 30 ml based on one gram of docetaxel.

According to the present invention, the crystal of the anhydrous crystalline docetaxel is prepared by adding an anti-solvent to a solution prepared by dissolving docetaxel in said organic solvent, in which the anti-solvent may be a C₅₋₇ alkane, such as pentane, hexane or heptane. Preferably, the anti-solvent is used in this reaction in an amount ranging from 1 to 5-fold by volume based on the volume of the organic solvent.

The anhydrous crystalline docetaxel thus formed may be isolated by collecting the crystal by filtration, and drying the crystal at a temperature ranging from 20 to 80° C. under a reduced pressure ranging from 0.1 to 10 torr. The anhydrous crystalline form of docetaxel thus obtained meets the purity requirement set by International Conference on Harmonization (ICH) Guideline which strictly limits the amount of residual solvents.

The anhydrous crystalline docetaxel of the present invention is stable and does not undergo any significant degradation during a long term storage, e.g., 7 days, at 40° C. under a relative humidity of 25% to 50%, in contrast to the docetaxel trihydrate which undergoes at least 50% dehydration under a comparable condition.

The method for the present invention, this provides for the first time high-purity docetaxel having a low 7-epimer content and a high storage stability.

The following Examples are intended to further illustrate the present invention without limiting its scope.

### Example 1

### Preparation of Anhydrous Crystalline Docetaxel A (1)

1 g of docetaxel (HPLC Purity: 99.7%) was dissolved in 20 ml of ethyl acetate at room temperature, and 30 ml of n-hexane was dropwisely added thereto. The mixture was stirred for 12 hrs at room temperature, and the precipitate formed was filtered, and dried at 60° C. under a pressure of 0.1 torr for 24 hrs, to obtain 0.95 g of an anhydrous crystalline form of docetaxel (Yield: 95%).

HPLC Purity: 99.8%;

The 7-epimer content: 0.03%;

The content of the title compound: 99.8%;

Melting point: 196˜203° C.; and

Residual solvent: ethyl acetate (63 ppm), n-hexane (5 ppm or less).

The powder X-ray diffraction spectrum of the anhydrous crystalline docetaxel thus prepared showed major peaks having a relative peak intensity of at least 20% (100×I/I₀: I; the intensity of the peak, and I₀; the intensity of the maximum peak), as shown in FIG. 2 and Table 1. The present inventors named the anhydrous crystalline docetaxel thus obtained “anhydrous crystalline docetaxel A”.

TABLE 1

| (2θ ± 0.1) | d | I/I₀ (%) |
|---|---|---|
| 4.6400 | 19.0296 | 502 |
| 8.0400 | 10.9896 | 1000 |
| 9.2400 | 9.5630 | 386 |
| 11.3400 | 7.7967 | 347 |
| 12.5400 | 7.0532 | 213 |
| 13.1800 | 6.7119 | 45 |
| 13.8600 | 6.3841 | 221 |
| 15.5200 | 5.7049 | 149 |
| 16.9200 | 5.2359 | 194 |
| 18.4800 | 4.7971 | 120 |
| 19.6400 | 4.5164 | 135 |
| 20.4000 | 4.3498 | 85 |
| 22.8200 | 3.8937 | 26 |
| 23.3600 | 3.8049 | 69 |
| 23.8200 | 3.7325 | 30 |
| 24.2000 | 3.6748 | 56 |
| 26.8400 | 3.3190 | 30 |
| 28.4000 | 3.1401 | 43 |
| 30.8200 | 2.8988 | 34 |
| 32.1000 | 2.7861 | 26 |

2θ: the diffraction angle,
d: distance between crystal facets, and
I/I₀: the relative peak intensity

### Example 2

### Preparation of Anhydrous Crystalline Docetaxel A (2)

1 g of docetaxel (HPLC Purity: 99.7%) was dissolved in 20 ml of methyl acetate at room temperature, and 30 ml of n-hexane was dropwisely added thereto. The mixture was stirred for 12 hrs at room temperature, and the precipitate formed was filtered, and dried at 60° C. under a pressure of 0.1 torr for 24 hrs, to obtain 0.94 g of the title compound (Yield: 94%).

HPLC Purity: 99.8%;

The 7-epimer content: 0.04%;

The content of the title compound: 99.7%;

Melting point: 194˜200° C.; and

Residual solvent: ethyl acetate (20 ppm or less), n-hexane (5 ppm or less).

### Example 3

### Preparation of Anhydrous Crystalline Docetaxel A (3)

1 g of docetaxel (HPLC Purity: 99.7%) was dissolved in 20 ml of dimethyl carbonate at room temperature, and 30 ml of n-hexane was dropwisely added thereto. The mixture was stirred for 12 hrs at room temperature, and the precipitate formed was filtered, and dried at 60° C. under a pressure of 0.1 torr for 24 hrs, to obtain 0.90 g of the title compound (Yield: 90%).

HPLC Purity: 99.8%;

The 7-epimer content: 0.02%;

The content of the title compound: 99.9%;

Melting point: 195˜203° C.; and

Residual solvent: dimethylcarbonate (185 ppm), n-hexane (5 ppm or less).

### Example 4

### Preparation of Anhydrous Crystalline Docetaxel B

1 g of docetaxel (HPLC Purity: 99.7%) was dissolved in a mixture of 10 ml of dichloromethane and 1 ml of methanol at room temperature, and 30 ml of n-hexane was dropwisely added thereto. The mixture was stirred for 12 hrs at room temperature, the precipitate formed was filtered, and dried at 60° C. under a pressure of 0.1 torr for 24 hrs, to obtain 0.98 g of another anhydrous crystalline form of docetaxel (Yield: 98%).

HPLC Purity: 99.8%;

The 7-epimer content: 0.02%;

The content of the title compound: 99.9%;

Melting point: 202˜209° C.; and

Residual solvent: dimethylcarbonate (185 ppm), n-hexane (5 ppm or less).

The powder X-ray diffraction spectrum of the anhydrous crystalline docetaxel thus prepared showed major peaks having a relative peak intensity of at least 20% (100×I/I₀), as shown in FIG. 3 and Table 2. The present inventors named the anhydrous crystalline docetaxel thus obtained “anhydrous crystalline docetaxel B”.

TABLE 2

| (2θ ± 0.1) | d | I/I₀ (%) |
|---|---|---|
| 4.8800 | 18.0931 | 819 |
| 7.6200 | 11.5929 | 24 |
| 8.3000 | 10.6440 | 24 |
| 9.2200 | 9.5841 | 121 |
| 9.7200 | 9.0918 | 1000 |
| 10.3800 | 8.5150 | 362 |
| 11.3000 | 7.8242 | 440 |
| 11.8800 | 7.4433 | 276 |
| 12.3400 | 7.1670 | 68 |
| 13.3400 | 6.6318 | 915 |
| 13.9600 | 6.3388 | 26 |
| 14.5600 | 6.0787 | 123 |
| 15.1400 | 5.8470 | 356 |
| 16.2200 | 5.4601 | 45 |
| 16.6200 | 5.3297 | 149 |
| 17.2800 | 5.1275 | 183 |
| 17.6600 | 5.0181 | 854 |
| 18.4000 | 4.8179 | 42 |
| 19.0200 | 4.6623 | 279 |
| 19.6200 | 4.5210 | 122 |
| 19.8600 | 4.4669 | 127 |
| 20.8600 | 4.2550 | 122 |
| 21.5000 | 4.1297 | 98 |
| 21.8600 | 4.0625 | 169 |
| 22.7400 | 3.9072 | 96 |
| 23.3200 | 3.8113 | 30 |
| 23.9800 | 3.7079 | 35 |
| 24.5800 | 3.6188 | 156 |
| 25.0000 | 3.5589 | 41 |
| 26.2000 | 3.3985 | 82 |
| 26.9800 | 3.3020 | 191 |
| 28.2200 | 3.1597 | 38 |
| 29.0600 | 3.0703 | 41 |
| 29.6000 | 3.0155 | 58 |
| 30.8200 | 2.8988 | 57 |
| 33.2400 | 2.6931 | 62 |
| 34.7200 | 2.5816 | 23 |
| 35.6200 | 2.5184 | 26 |

2θ: the diffraction angle,
d: distance between crystal facets, and
I/I₀: the relative peak intensity

### Example 5

### Preparation of Anhydrous Crystalline Docetaxel C

1 g of docetaxel (HPLC Purity: 99.7%) was dissolved in a mixture of 10 ml of dichloromethane and 1 ml of acetonitrile at room temperature, and 30 ml of n-hexane was dropwisely added thereto. The mixture was stirred for 12 hrs at room temperature, and the precipitate formed was filtered, and dried at 60° C. under a pressure of 0.1 torr for 24 hrs, to obtain 0.98 g of still another anhydrous crystalline form of docetaxel (Yield: 98%).

HPLC Purity: 99.8%;

The 7-epimer content: 0.03%;

The content of the title compound: 99.9%;

Melting point: 198˜206° C.; and

Residual solvent: acetonitrile (50 ppm), n-hexane (5 ppm or less).

The powder X-ray diffraction spectrum of the anhydrous crystalline docetaxel thus prepared showed major peaks having a relative peak intensity of at least 20% (100×I/I₀), as shown in FIG. 4 and Table 3. The present inventors named the anhydrous crystalline docetaxel thus obtained “anhydrous crystalline docetaxel C”.

TABLE 3

| (2θ ± 0.1) | d | I/I₀ (%) |
|---|---|---|
| 4.6200 | 19.1095 | 360 |
| 7.1800 | 12.3017 | 25 |
| 8.2200 | 10.7470 | 190 |
| 9.2000 | 9.6044 | 1000 |
| 10.6400 | 8.3078 | 431 |
| 11.4400 | 7.7286 | 167 |
| 12.4200 | 7.1210 | 205 |
| 13.2600 | 6.6716 | 49 |
| 13.8000 | 6.4115 | 249 |
| 14.2000 | 6.2321 | 73 |
| 15.2800 | 5.7938 | 206 |
| 17.2800 | 5.1275 | 214 |
| 17.7600 | 4.9910 | 54 |
| 18.4600 | 4.8024 | 138 |
| 19.4400 | 4.5625 | 30 |
| 20.6200 | 4.3039 | 55 |
| 21.0800 | 4.2111 | 32 |
| 21.5600 | 4.1183 | 46 |
| 21.0800 | 4.0625 | 111 |
| 22.3000 | 3.9833 | 46 |
| 23.0400 | 3.8570 | 45 |
| 23.5200 | 3.7793 | 32 |
| 24.8800 | 3.5758 | 24 |
| 26.2600 | 3.3909 | 27 |

2θ: the diffraction angle,
d: distance between crystal facets, and
I/I₀: the relative peak intensity

### Example 6

### Preparation of Anhydrous Crystalline Docetaxel D

1 g of anhydrous crystalline docetaxel A (HPLC Purity: 99.7%) prepared in Example 1 was dissolved in 30 ml of diethyl ether, and stirred for 12 hrs, and then 20 ml of n-hexane was dropwisely added thereto. The mixture was stirred for 12 hrs at room temperature, and the precipitate formed was filtered, and dried at 60° C. under a pressure of 0.1 torr for 24 hrs, to obtain 0.88 g of a further anhydrous crystalline form of docetaxel (Yield: 88%).

HPLC Purity: 99.8%;

The 7-epimer content: 0.04%;

The content of the title compound: 99.7%;

Melting point: 192˜200° C.; and

Residual solvent: diethyl ether (180 ppm), n-hexane (5 ppm or less).

The powder X-ray diffraction spectrum of the anhydrous crystalline docetaxel thus prepared showed major peaks having a relative peak intensity of at least 20% (100×I/I₀), as shown in FIG. 5 and Table 4. The present inventors named the anhydrous crystalline docetaxel thus obtained “anhydrous crystalline docetaxel D”.

TABLE 4

| (2θ ± 0.1) | d | I/I₀ (%) |
|---|---|---|
| 4.0600 | 21.7439 | 127 |
| 4.8200 | 18.3184 | 256 |
| 5.9800 | 14.7667 | 39 |
| 7.5800 | 11.6537 | 380 |
| 8.2000 | 10.7736 | 1000 |
| 9.8400 | 8.9816 | 163 |
| 11.4400 | 7.7286 | 105 |
| 12.7600 | 6.9320 | 91 |
| 13.6200 | 6.4961 | 93 |
| 14.1600 | 6.2497 | 65 |
| 15.7800 | 5.6114 | 44 |
| 16.9800 | 5.2173 | 90 |
| 18.4800 | 4.7971 | 49 |
| 19.1800 | 4.4637 | 50 |
| 19.6000 | 4.5255 | 84 |
| 19.9000 | 4.4580 | 59d |

2θ: the diffraction angle,
d: distance between crystal facets, and
I/I₀: the relative peak intensity

### Test Example 1

### Stability Under a High Temperature/Humidity Condition

The long-term storage stabilities of anhydrous crystalline forms of docetaxel prepared in Examples 1, 4, 5, and 6, respectively were compared with that of docetaxel trihydrate prepared according to the method of U.S. Pat. No. 6,022,985 by subjecting sample thereof to aging under a high temperature/humidity condition (60±2° C.; 75±5% relative humidity). The amounts of the original compound in each sample after 1, 2, 4 and 8 weeks were determined by high performance liquid chromatography (HPLC). The purities of each sample were shown in Table 5.

TABLE 5

| | | | Purity | Purity | Purity | Purity |
|---|---|---|---|---|---|---|
| | | Initial | after | after | after | after |
| Compound | | purity | 1 week | 2 weeks | 4 weeks | 8 weeks |
| The | Example 1 | 99.8% | 99.8% | 99.8% | 99.7% | 99.6% |
| anhydrous | (Form A) | | | | | |
| crystaline | Example 4 | 99.8% | 99.8% | 99.7% | 99.6% | 99.5% |
| docetaxel | (Form B) | | | | | |
| | Example 5 | 99.7% | 99.7% | 99.7% | 99.6% | 99.6% |
| | (Form C) | | | | | |
| | Example 6 | 99.7% | 99.7% | 99.7% | 99.6% | 99.5% |
| | (Form D) | | | | | |

| | | | | | |
|---|---|---|---|---|---|
| Docetaxel trihydrate | 99.4% | 99.3% | 99.0% | 98.7% | — |

As shown in Table 5, the anhydrous crystalline forms of docetaxel of the present invention were stable for 8 weeks under a high temperature/humidity condition, in contrast to the docetaxel trihydrate which undergoes rapid degradation under the same condition. The above result shows that the anhydrous crystalline forms of docetaxel of the present invention are more stable than docetaxel trihydrate prepared by the conventional method.

While the invention has been described with respect to the above specific embodiments, it should be recognized that various modifications and changes of the invention also fall within the scope of the present invention defined by the claims that follow.

1.-11. (canceled)

12. An anhydrous crystalline form of docetaxel of formula (I), whose X-ray diffraction spectrum shows major peaks at 2θ values of 4.88, 9.22, 9.72, 10.38, 11.30, 11.88, 13.34, 14.56, 15.14, 16.62, 17.28, 17.66, 19.02, 19.62, 19.86, 20.86, 21.86, 24.58, and 26.98; and which contains 0.1% or less of the 7-epimer thereof: 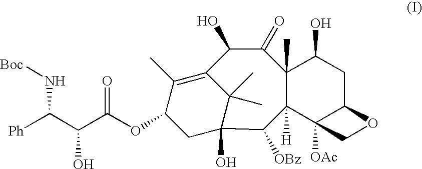
wherein,
Ph is phenyl;
Ac is acetyl;
Bz is benzoyl; and
Boc is t-butoxycarbonyl.

13. An anhydrous crystalline form of docetaxel of formula (I), whose X-ray diffraction spectrum shows major peaks at 2θ values of 4.62, 8.22, 9.20, 10.64, 11.44, 12.42, 13.80, 14.20, 15.28, 17.28, 18.46, 20.62, and 21.86; and which contains 0.1% or less of the 7-epimer thereof: 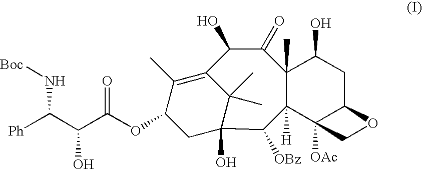
wherein,
Ph is phenyl;
Ac is acetyl;
Bz is benzoyl; and
Boc is t-butoxycarbonyl.

14. An anhydrous crystalline form of docetaxel of formula (I), whose X-ray diffraction spectrum shows major peaks at 2θ values of 4.06, 4.82, 7.58, 8.20, 9.84, 11.44, 12.76, 13.62, 14.16, 16.98, 19.18, 19.60, and 19.90; and which contains 0.1% or less of the 7-epimer thereof: 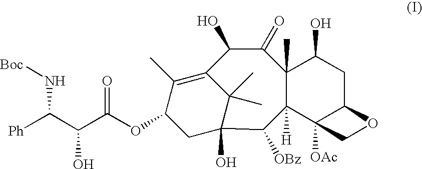
wherein,
Ph is phenyl;
Ac is acetyl;
Bz is benzoyl; and
Boc is t-butoxycarbonyl.

15. A method of preparing the anhydrous crystalline form of docetaxel of claim 12 which comprises the steps of:
(i) dissolving docetaxel in a dichloromethane-methanol mixture as an organic solvent;
(ii) adding a hexane to the resulting solution; and
(iii) recovering the resulting crystals.

16. method of preparing the anhydrous crystalline form of docetaxel of claim 13 which comprises the steps of:
(i) dissolving docetaxel in a dichloromethane-acetonitrile mixture as an organic solvent;
(ii) adding a hexane to the resulting solution; and
(iii) recovering the resulting crystals.

17. A method of preparing the anhydrous crystalline form of docetaxel of claim 14 which comprises the steps of:
(i) dissolving an anhydrous crystalline form of docetaxel of formula (I), whose X-ray diffraction spectrum shows major peaks at 2θ values of 4.64, 8.04, 9.24, 11.34, 12.54, 13.86, 15.52, 16.92, 18.48, 19.64, 20.40, 23.36, and 24.20 in a dimethyl ether as an organic solvent;
(ii) adding a hexane to the resulting solution; and
(iii) recovering the resulting crystals: 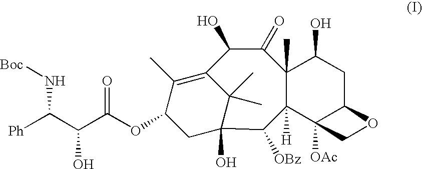
wherein,
Ph is phenyl;
Ac is acetyl;
Bz is benzoyl; and
Boc is t-butoxycarbonyl.

18. The method according to claim 15, wherein step (iii) comprises collecting the crystals by filtration, and drying the crystals at a temperature ranging from 20 to 80° C. under a reduced pressure ranging from 0.1 to 10 torr.

19. The method according to claim 15, wherein the organic solvent is used in an amount ranging from 5 to 30 ml based on one gram of docetaxel.

20. The method according to claim 15, wherein the hexane is used in an amount ranging from 1 to 5-fold by volume based on the volume of the organic solvent.

21. The method according to claim 16, wherein step (iii) comprises collecting the crystals by filtration, and drying the crystals at a temperature ranging from 20 to 80° C. under a reduced pressure ranging from 0.1 to 10 torr.

22. The method according to claim 16, wherein the organic solvent is used in an amount ranging from 5 to 30 ml based on one gram of docetaxel.

23. The method according to claim 16, wherein the hexane is used in an amount ranging from 1 to 5-fold by volume based on the volume of the organic solvent.

24. The method according to claim 17, wherein step (iii) comprises collecting the crystals by filtration, and drying the crystals at a temperature ranging from 20 to 80° C. under a reduced pressure ranging from 0.1 to 10 torr.

25. The method according to claim 17, wherein the organic solvent is used in an amount ranging from 5 to 30 ml based on one gram of docetaxel.

26. The method according to claim 17, wherein the hexane is used in an amount ranging from 1 to 5-fold by volume based on the volume of the organic solvent.
